(19) United States
(12) Patent Application Publication
(10) Pub. No.: US 2008/0274062 A1
(43) Pub. Date: Nov. 6, 2008

(54) Glycyrrhizic acid compounds as a foamer in chemically-derived surfactant-free dentifrice

(76) Inventors: Chantal Bergeron, Kennebunk, ME (US); Julie Venell, Acton, ME (US)

Correspondence Address: Kevin M. Farrell;Pierce Atwood, Suite 350, One New Hampshire Avenue, Portsmouth, NH, 03801, US

(21) Appl. No.: 11/799,523

(22) Filed: May 2, 2007

(51) Int. Cl. A61K 8/00; A61K 8/21; A61Q 11/00

(52) U.S. Cl. 424/49; 424/52

(57) ABSTRACT

The present invention relates to dentifrices in general and dentifrices free of chemically-derived foaming agents in particular. Particularly, the present invention relates to an all-natural dentifrice (e.g., toothpaste) comprising a concentration of glycyrrhizic acid that is suitable to work as a foaming agent. Even more particularly, the present invention relates to an all-natural dentifrice (e.g., toothpaste) that does not contain one or more of sodium lauryl sulfate, sodium lauroyl sarcosinate, cocomidopropyl betaine, sodium methyl cocoyl taurate, sodium cocoyl glutamate, poloxamer or any other chemically-derived foaming agent.

### BACKGROUND

Sodium Lauryl Sulfate (SLS, also known as sodium dodecyl sulfate or SDS) is a well known and widely used ionic surfactant. It is used in personal care and household products such as toothpastes, shampoos, shaving creams and bubble baths for its emulsifying property and its ability to create a foam or lather.

Other chemically-derived foamers are also known in the art and have been used with oral care products. For example, poloxamers, have been used as foamers but are lower in foaming ability than SLS. Poloxamers are the nonionic block copolymers composed of a central hydrophobic chain of polyoxypropylene (poly-propylene oxide) flanked by two hydrophilic chains of polyoxyethylene (poly-ethylene oxide). Because the lengths of the polymer blocks can be customized, many different poloxamers exist that have slightly different properties.

Nonionic cellulose ethers (NCE) have also been used either alone in combination with poloxamers as a foaming agent.

However, some consumers dislike using oral care products with chemically-derived ingredients. For example, for people suffering from canker sores the use of SLS-free toothpaste is recommended. Additionally, these agents tend to have an unpleasant taste that has to be masked with high quantities of sweeteners or flavorings.

Other chemically-derived foamers are also available but have similar consumer acceptance issues. Some examples of these are sodium lauryl sarcosinate, cocomidopropyl betaine, sodium methyl cocoyl taurate and sodium cocoyl glutamate.

Thus, what is needed is a dentifrice that is free of chemically-derived foaming agents but provides for the consumer the same or similar mouth-feel and foaming characteristic as, for example, an SLS-containing oral care product.

### SUMMARY OF THE INVENTION

The present invention provides, in one aspect, a dentifrice that is free of SLS and similar chemically-derived foamers and foaming agents. In this regard, the present invention provides for a dentifrice that contains only natural ingredients. In another embodiment, the present invention relates to a dentifrice that is free of chemically-derived foaming agents. In yet another aspect, the present invention provides for all natural dentifrice comprising a concentration of glycyrrhizic acid or glycyrrhizic acid compound sufficient to provide foaming activity. Glycyrrhizic acid (glycyrrhizin) has been used as a sweetener in various products in place of table sugar, sucrose or artificial sweeteners. It is isolated from liquorice (licorice) root and is 30-50 times as potent as sucrose as a sweetener. However, use of glycyrrhizic acid as a foaming agent is not known in the prior art. In particular, use of glycyrrhizic acid in a dentifrice at concentrations and in formulations permitting it to function as a foaming agent was unknown until the conception of the present invention. In a preferred embodiment, the dentifrice of the present invention is a toothpaste or gel.

Other dental products have used glycyrrhizic acid and various derivatives of glycyrrhizic acid (as, for example, a sweetener) in formulations or at concentrations too low to aid in the foaming properties necessary for effective cleaning by a dentifrice.

For example, U.S. Pat. No. 5,496,541 to Cutler discloses a dentifrice paste comprising a foaming agent consisting of three ingredients: a poloxamer, an anionic polysaccharide and nonionic cellulose ether. Cutler discloses incorporation into this dentifrice paste 0.4% by weight of dipotassium glycyrrhizinate and into a dentifrice gel 0.2% by weight of disodium glycyrrhizinate as anti-adherents. Cutler also teaches the use of glycyrrhizin as a sweetener. However, Cutler teaches against the use of glycyrrhizin as a foamer as in the present invention when it is stated that the level of glycyrrhizic acid used in the specification does not “materially affect foam production” (U.S. Pat. No. 5,496,541, column 5, line 65-column 6, line 3). Cutler may not see foaming action associated with glycyrrhizic acid because other ingredients comprising his toothpaste may lessen the foam making ability of glycyrrhizic acid. In any event, Cutler does not disclose a composition or formulation wherein a glycyrrhizic compound functions as a foaming agent nor does he disclose a dentifrice free of chemically-derived foamers.

Goultschin, et al., (J. Clin. Periodontol., 18:210-212, 1991), reported that the incorporation of 0.25-0.50% glycyrrhizin as an anti-plaque agent in toothpaste was not effective in the removal or prevention of plaque build up when compared to control toothpastes. The toothpastes made for the study contained SLS as the foaming agent. Goultschin, et al., do not disclose a composition wherein a glycyrrhizic compound functions as a foaming agent nor do they disclose a dentifrice free of chemically-derived foamers.

Still others have used liquorice extract (which contains glycyrrhizic acid) as an antibacterial, antiviral or anti-inflammatory agent. For example, U.S. Pat. No. 6,319,523 to Zhou describes an oral care composition comprising 0.001% w/v liquorice extract in combination with 0.05% w/v of a polyphenol composition and about 0.01% w/v of a mogroside composition as an anti-bacterial. Mogrosides are triterpene-glycosides that are typically isolated from the fruit (called Monk's fruit) of the plant Siraitia grosvenorii and related species. This publication does not disclose the use of glycyrrhizic acid as a foaming agent or at concentrations nor in formulations where it could function as a foaming agent.

U.S. Pat. No. 6,881,427 to Mayne, et al., discloses a topical anti-inflammatory composition containing 0.001 to 1.5% w/w of liquorice extract in combination with linseed extract (licorice extract typically contains between 10-35% glycyrrhizin of the extracted solids). This publication does not disclose the use of glycyrrhizic acid in an oral care product, as a foaming agent or at concentrations nor in formulations where it could function as a foaming agent.

U.S. Pat. No. 4,278,657 to Tezuka, et al., discloses a skin cream composition containing glycyrrhizic acid in combination with water-soluble polysaccharides such as pectin or locust bean gum for use as an emulsifier (an emulsion defined in the art as a suspension of small globules of a first liquid in a second liquid with which the first will not mix. An emulsifier aids in creating the suspension by encasing the first liquid). This publication does not disclose the use of glycyrrhizic acid in an oral care product, as a foaming agent or at concentrations nor in formulations where it could function as a foaming agent.

Chemically, glycyrrhizin is a triterpenoid glycosidic saponin with the systematic name (3-beta,20-beta)-20-carboxy-11-oxo-30-norolean-12-en-3-yl 2-O-beta-D-glucopyranuronosyl-alpha-D-glucopyranosiduronic acid. The acid form is not particularly water-soluble, but its salt is soluble in water at pH greater than 4.5.

Thus, the present invention is related to toothpastes and other similar dentifrice products that utilize glycyrrhizic acid and glycyrrhizic acid compounds as a foaming agent. In another regards, the present invention is related to toothpastes and other dentifrices that exclude chemically-derived foaming agents. In this regard, the concentration of glycyrrhizic acid used in the present invention is at least approximately 0.1% percent. In a more preferred embodiment, the concentration of glycyrrhizic acid is between approximately 0.1-5.0% or 0.1-2.0% percent. In a most preferred embodiment, the concentration of glycyrrhizic acid when used in the present invention is about 0.2-0.4% percent.

In a preferred embodiment, the dentifrice of the present invention does not comprise poloxamers or SLS. In another embodiment, the dentifrice of the present invention consists of only one or more glycyrrhizin compounds as the foaming agent.

### DESCRIPTION OF THE PREFERRED EMBODIMENTS

The present invention relates to a dentifrice comprising a concentration of glycyrrhizic acid (in the form of one or more of glycyrrhizic acid salts or compounds or one or more glycyrrhizic acid containing compounds) that is suitable to work as a foaming agent without the addition of other foamers such as chemically-derived compounds.

In the present invention, the term “chemically-derived” means a substance or compound not found in nature or a chemical compound found in nature that has had its chemical structure modified by a chemical process effected by artificial means either during or after being extracted from the natural source. A substance or compound that naturally changes structure during or after extraction and/or isolation, e.g., by binding or giving up sodium, potassium, water groups, or the like, without artificial chemical intervention, is not considered to be chemically-derived. A composition free of “chemically-derived” substances refers to a composition that does not contain any chemically-derived substances or contains only minute (i.e., trace) quantities of chemically-derived substances.

In the present invention, “dentifrice” is defined as a substance such as a paste, gel, powder, liquid or other preparation for cleaning the teeth that is usually, but not always, applied with a toothbrush or other dental implement. Other suitable definitions of dentifrice may be known in the art.

In the present invention, “foam” is defined as a collection of minute bubbles creating a frothy substance. A “foamer” or “foaming agent” is one or more agents that are capable of causing foam, usually when agitated such as with a brush or similar device. Other suitable definitions of foam or foamer may be known in the art.

Most persons expect a certain mouth feel from their toothpaste (and other dentifrices) when brushing or otherwise cleaning their teeth. Toothpastes typically generate this mouth feel by adding one or more agents that function as a foaming agent or foamer. In addition to generating a pleasant mouth feel, the foaming agent used in toothpastes also works to disperse the active ingredients found in the toothpaste in the user's mouth. (This agent also creates the “foaming at the mouth” look that is typical of most toothpaste when used.) Foaming agents are also used in toothpastes that are more commonly referred to as gels. The most common foamer used in toothpastes today is SLS.

Even so, many people prefer to use products without artificial ingredients sometimes called “all-natural” products. Although there are several available all-natural toothpastes on the market, none of them utilize glycyrrhizic acid as a foamer. Some such “all-natural” toothpastes use, for example, cellulose gum (Methocel®, Dow Chemical Corp.) as foamers. However, these agents are not considered to be all-natural and are not considered to be non-chemically-derived. Additionally, even these agents are considered to be chemically-derived substances. Many people do not like to use chemically-derived substances. One reason is that they are concerned that the agents used in the modification of the substance (which may be toxic or otherwise harmful in larger quantities) may be carried through to the end product or they want to use all-natural products

In a preferred embodiment, the dentifrice of the present invention does not contain, for example, one or more of sodium lauryl sulfate, sodium lauroyl sarcosinate, cocomidopropyl betaine, sodium methyl cocoyl taurate, sodium cocoyl glutamate, poloxamer(s), anionic polysaccharides or non-ionic polysaccharides. In another preferred embodiment, the present invention does not comprise a chemically-derived foaming agent.

In another embodiment, the dentifrice of the present invention comprises one or more glycyrrhizic acid compounds (e.g., salts) as foaming agents. In yet another embodiment, the dentifrice of the present invention comprises only one or more glycyrrhizic acid compounds foaming agents. In still yet another embodiment, the foaming agent of the dentifrice of the present invention consists of one or more glycyrrhizic acid compounds.

Toothpastes (and other similar dentifrices such as tooth gels, toothpowders and denture cleansing liquids and pastes, and the like) will generally comprise a binder or thickening agent. Binders suitable for use herein include carboxyvinyl polymers, carrageenan (preferred), hydroxyethyl cellulose and water-soluble salts of cellulose ethers such as sodium carboxymethyl cellulose and sodium carboxymethyl hydroxyethyl cellulose. Natural gums such as gum karaya (an extract of sterculia trees that is used as a thickener, emulsifier and laxative in foods and as a denture adhesive), xanthan gum (preferred embodiment; produced by a biotechnological process involving fermentation of glucose or sucrose by the Xanthomonas campestris bacterium), gum Arabic (from the acacia tree), and gum tragacanth (from a groups of legumes collectively known as “goat's thorn” or “locoweed”) can also be used. Colloidal magnesium aluminum silicate or finely divided hydrated silica can be used as part of the thickening agent to further improve texture. Binders/thickening agents can be used in an amount from about 0.1% to about 15.0%, preferably from about 1.0% to about 12.0% by weight of the total composition.

In many dentifrices, the active ingredients are fluoride (for cavity prevention; e.g., sodium monofluorophosphate [NaMFP], sodium fluoride and stannous fluoride) and potassium nitrate (as an aid for people with sensitive teeth). Their active levels for use as anti-cavity and sensitivity agents are described below and in their respective FDA monographs, which are incorporated herein by reference.

Fluorides are used in dentifrices, and are recognized by the American Dental Association, to effectively and safely prevent tooth decay. One or more forms of fluoride may be used in the dentifrices of the present invention. Dentifrices comprising calcium carbonate preferably use sodium monofluorophosphate. The preferred concentration for NaMFP is approximately 0.13-0.15% % w/v fluoride ion and for NaF it is approximately 0.243%.

For people with sensitive teeth, the formulation may contain an agent for desensitizing pain. A preferred ingredient for this purpose is potassium nitrate. A preferred concentration range of potassium nitrate for use in the present invention is between 3 and 7%. A more preferred range is between 4.5-5.5%.

One active ingredient suitable for the treatment of gingivitis is zinc citrate. A preferred concentration is between 1 and 3% and a preferred concentration is about 2%.

Abrasives suitable for use in the dentifrices of the present invention are, for example, one or more of calcium carbonate, baking soda or hydrated silica at a concentration of between 10-70%, 12-50% or 15-40%.

The dentifrice compositions of the present invention will typically comprise one or more sweeteners and flavorings. Examples of suitable sweeteners and flavorings for use in dentifrices (e.g., toothpastes) are discussed herein. Preferred flavorings are natural flavorings such as peppermint, spearmint, apricot, fennel, clove, black currant, orange, grape, watermelon, mango, wintergreen, ginger, strawberry and cinnamon, etc.

The flavors used in the dentifrices of the present invention may be added as oils or as spray dried flavors. Spray drying is a technique that generates powdered oil. Spray dried flavors are made by, for example, spraying the oil(s) on carrier substances like Arabic gum, maltodextrin or starch and then drying with a spray dryer. Spray drying is discussed in more detail elsewhere in this specification. Spray dried flavors (spray dried flavor compositions) used in the present invention rehydrate, for example, upon use. Additionally, flavors may be solubilized into carriers such as glycerin and they can also be extracted in glycerin (i.e., glycerin-based flavors). In another embodiment, water soluble flavors are contemplated for use in the present invention. Flavors are added in concentrations and combinations necessary to generate the desired taste.

In a preferred embodiment, the present invention contemplates the use of water soluble flavors (e.g., spray dried and in glycerin carriers). It has been found by the inventors that the use of water soluble flavors aids the foaming effected by the glycyrrhizic acid. Thus, is a preferred embodiment of the present invention, the foaming agent comprises one or more glycyrrhizic acid compounds and one or more water soluble flavors (e.g., spray dried and in glycerin).

pH balancing agents (e.g., citric acid, NaOH) as well as surfactants (e.g., those known in the art), preservatives (e.g., benzoic acid, sorbic acid), stabilizers, etc., are also often used in dentifrice compositions, examples of which are also provided herein or known in the art. Also, the dentifrice of the present invention may comprise, for example, botanicals like aloe, calendula and myrrh.

It is also desirable to include some humectant material in toothpaste to keep the composition from hardening upon exposure to air. A preferred but non-limiting humectant for use in the present invention is one or more of glycerin and sorbitol. Certain humectants can also impart a desirable sweetness to toothpaste compositions.

Liquid dentifrice can also contain a quantity of humectant. Humectants promote the retention of moister. Suitable humectants are well known in the art. For example, glycerin and sorbitol are two exemplary humectants. When present, humectants generally represent from about 10% to about 70%, by weight of the compositions of the invention.

Toothpowders may make use of many of the same ingredients as toothpastes except that they must be mixed in a dried state (i.e., dry milled) or mixed as a liquid composition and then dried via, for example, various known spray drying techniques. Spray drying is described as when a liquid form of a composition is sprayed as a mist into a hot, dry chamber wherein the aqueous portion of the mist is evaporated by the dry heat of the chamber leaving only the dry constituents of the composition in a powdered form. The powdered form of the composition has a moisture content of between approximately 0.1% to 5.0%. Toothpowders then rehydrate upon use either by the addition of water (e.g., with water applied by the user) at the time of use or by the user's saliva.

Denture cleanser compositions of the invention can additionally include one or more cleaning agents. Non-limiting examples include oxidants, which remove stains and whiten the denture teeth (for example, an alkaline perborate such as sodium perborate and potassium monopersulfate), effervescence generators, which provide mechanical cleaning action to loosen particles from the denture (for example, perborate such as sodium perborate and potassium monopersulfate and/or carbonate such as sodium bicarbonate), chelating agents (for example, EDTA, citric acid, hypochlorite and dilute hypochlorite [used in liquid denture cleaner, for example]), etc., which are known to those practiced in the art. Also, proteolytic enzyme-containing cleaning agents designed to break down mucin deposits on dentures and to break down food protein in plaque may be included in the denture cleaners of the present invention. Additionally, detergents (which clean by removing particles broken down by the above mentioned ingredients), color and fragrance agents may be present as well.

Glycyrrhizic acid may be extracted from the root of many different species of legume-like pants from the genus Glycyrrhiza and more particularly from the species Glycyrrhiza glabra. The plants are native to southern Europe and parts of Asia. The related Chinese liquorice (G. uralensis), which is used extensively in traditional Chinese medicine, contains glycyrrhizic acid in much greater concentration than its Western counterpart. Although there are many different sources for glycyrrhizic acid, including those listed here, the present invention is not limited to any specific source.

Glycyrrhizic acid is a constituent of liquorice extract. Liquorice is traditionally extracted by the use of organic solvents or with water and then the extract is acidified and/or ethanol is added to aid in the extraction process. The preparation of glycyrrhizic acid (as, for example, ammonium or potassium salts of glycyrrhizic acid) is well know and readily available (e.g., by FLUKA AG). The present invention is not limited to any particular glycyrrhizic acid or glycyrrhizin compound and may be selected from one or more of, for example, glycyrrhizic acid and salts thereof such as monosodium glycyrrhizinate, monopotassium glycyrrhizinate, disodium glycyrrhizinate, dipotassium glycyrrhizinate, trisodium glycyrrhizinate and tripotassium glycyrrhizinate. Among these compounds, glycyrrhizic acid, monosodium glycyrrhizinate, monopotassium glycyrrhizinate, disodium glycyrrhizinate, dipotassium glycyrrhizinate, trisodium glycyrrhizinate and tripotassium glycyrrhizinate are preferred. The foregoing glycyrrhizic compounds may be used alone or in combination in the present invention.

The present invention is not limited to the source or method of extraction of glycyrrhizic acid. In a non-limiting example, glycyrrhizic acid and salts thereof can be obtained in the following manner: Dried licorice roots were cut into small pieces and extracted with water in the cold. Ethanol was added to this aqueous extract and the resulting precipitate was filtered off. After addition of a mineral acid to the filtrate, the resulting precipitate was collected by filtration and washed with water to remove any residual mineral acid. The product thus obtained was glycyrrhizic acid. This glycyrrhizic acid was dissolved in an aqueous solution containing a base such as, but not limited to sodium hydroxide or potassium hydroxide and then evaporated to dryness. The residue was recrystallized from a solvent such as acetic acid or alcohol to obtain a monobasic salt of glycyrrhizic acid. Dibasic and tribasic salts of glycyrrhizic acid can also be obtained by dissolving the monobasic salt in the stoichiometric amount of the aforesaid aqueous solution containing a base and then evaporating it to dryness. Other methods are known in the art and incorporated herein.

Glycyrrhizic acid is a naturally occurring triterpenoid saponin, which can be found in extracts of roots and rhizomes of the licorice plant Glycyrrhiza glabra, for example, together with a number of other substance including triterpenoids, polyphenols, polysaccharides, essential oils and flavonoids. The crude dried aqueous extracts (also known as “block licorice”) may contain 4-25% glycyrrhizic acid in the form of calcium, magnesium and potassium salts (Wang, et al., 2000; Stormer, et al., 1993a: EFFA, 2001).

Licorice (glycyrrhiza) root is the dried and ground rhizome and root portions of Glycyrrhiza glabra or other species of Glycyrrhiza. Licorice extract is that portion of the licorice root that is, after maceration, extracted by, for example, boiling water. The extract can be further purified by filtration and by treatment with acids and ethyl alcohol. Licorice extract is sold as a liquid, paste (“block”), or dried powder.

### EXPERIMENTAL

### Example 1

In this example, various concentrations of glycyrrhizic acid were used to determine the concentration of glycyrrhizic acid necessary to function as a foamer. As a sweetener, glycyrrhizic acid is typically used at a concentration of 0.001-0.01%. As can be seen in Table 1, the concentrations of glycyrrhizic acid used were 0.01%, 0.10% and 0.30%. Ten grams of toothpaste were diluted in water to reach 50 ml. Fifty milliliters of test solution were placed in a 100 ml graduated cylinder. The samples were then inverted 10 times and observed for volume and density of foam formed. Samples were examined at time zero after inverting and at 5, 10 and 15 minutes.

The test samples with 0.01% glycyrrhizic produced only 1 ml of foam consisting of mostly large bubbles and which was mainly around the glass wall of the cylinder due to surface tension. The test samples with 0.10% glycyrrhizic acid produced 3 ml of dense foam covering the surface of the sample and comprising mostly small bubbles. The bubbles lasted for more than 15 minutes. The test samples with 0.30% glycyrrhizic acid produced between 5 and 7 ml of dense foam covering the surface of the sample and comprising mostly small bubbles. The bubbles lasted for more than 15 minutes.

TABLE 1
Comparison of the foaming effect of glycyrrhizic acid used at
concentrations suitable for use as a sweetener (0.01%) or at
concentrations suitable for use as a foamer (0.10 and 0.30%).

| | Concentration of Glycyrrhizic Acid | | | | | |
|---|---|---|---|---|---|---|
| | | | | 0.10% | 0.30% | 0.30% |
| | 0.01% A* | 0.01% B | 0.10% A | B | A | B |
| Initial | 1 | 1 | 3 | 3 | 6 | 7 |
| 5 min | 1 | 1 | 3 | 3 | 6 | 7 |
| 10 min | 1 | 1 | 3 | 2 | 5 | 6 |
| 15 min | 1 | 1 | 3 | 2 | 5 | 6 |

*A and B represent duplicates.

We claim:

1. A dentifrice comprising a foaming agent, said foaming agent comprising one or more glycyrrhizic acid compounds wherein the glycyrrhizic acid content of said glycyrrhizic acid compounds is at a concentration of between approximately 0.1% and 5.0% w/w of said dentifrice and wherein said dentifrice does not comprise poloxamer or SLS.

2. The dentifrice of claim 1, wherein said glycyrrhizic acid is at a concentration of between approximately 0.1% and 2.0% w/w of said dentifrice.

3. The dentifrice of claim 1, wherein said glycyrrhizic acid is at a concentration of between approximately 0.2% and 0.4% w/w of said dentifrice.

4. The dentifrice of claim 1, wherein the foaming agent consists of one or more glycyrrhizic acid compounds.

5. The dentifrice of claim 1, wherein the glycyrrhizic acid compound is at least one compound selected from the group consisting of glycyrrhizic acid and its salts, monosodium glycyrrhizinate, monopotassium glycyrrhizinate, disodium glycyrrhizinate, dipotassium glycyrrhizinate, trisodium glycyrrhizinate and tripotassium glycyrrhizinate.

6. The dentifrice of claim 1, wherein said dentifrice additionally comprises water, one or more abrasives, one or more humectants, one or more flavors and one or more thickeners.

7. The dentifrice of claim 6, wherein said one or more abrasives are selected from a group consisting of silica, calcium carbonate or baking soda (sodium bicarbonate).

8. The dentifrice of claim 6, wherein said one or more humectants are selected from a group consisting of glycerin or sorbitol at a total concentration of between approximately 10-70%.

9. The dentifrice of claim 6, wherein said one or more flavors are selected from a group consisting of peppermint, spearmint, strawberry, apricot, clove, ginger, wintergreen, mango, fennel, orange, black currant, watermelon or cinnamon.

10. The dentifrice of claim 6, wherein said one or more thickening agents are selected from a group consisting of carboxyvinyl polymers, carrageenan, hydroxyethyl cellulose, water soluble salts of cellulose ethers, gum karaya, xanthan gum, gum Arabic, gum tragacanth, colloidal magnesium silicate, finely divided hydrated silica or hectorite clay.

11. The dentifrice of claim 10, wherein said one or more thickening/binding agents are at a concentration of between approximately 0.1-15.0%.

12. The dentifrice of claim 6, wherein said abrasive is selected from one or more of calcium carbonate, sodium bicarbonate or silica at a concentration of between approximately 10-50% of the dentifrice.

13. The dentifrice of claim 1, wherein said toothpaste additionally comprises water, calcium carbonate, one or more of sodium bicarbonate or silica, glycerin, xylitol, sorbitol, flavor and carrageenan.

14. The dentifrice of claim 1, wherein said toothpaste additionally comprises a fluoride treatment.

15. The dentifrice of claim 14, wherein said fluoride is provided as one or more of sodium fluoride, sodium monofluorophosphate or stannous fluoride.

16. The dentifrice of claim 1, wherein said dentifrice does not contain one or more of sodium lauryl sulfate, sodium lauroyl sarcosinate, cocomidopropyl betaine, sodium methyl cocoyl taurate, sodium cocoyl glutamate or poloxamer.

17. The dentifrice of claim 1, wherein said dentifrice is selected from a group consisting of toothpaste, toothpaste gels, tooth power, denture cleaners or liquid toothpaste.

18. The dentifrice of claim 1, wherein it additionally comprises potassium nitrate.

19. The dentifrice of claim 18, wherein said potassium nitrate is at a concentration of approximately 5% by weight.

20. The dentifrice of claim 1, wherein it additionally comprises zinc citrate.

21. The dentifrice of claim 20, wherein said zinc citrate is at a concentration of approximately 2% by weight.

22. The dentifrice of claim 1, wherein said dentifrice additionally comprises a water soluble flavoring.

23. The water soluble flavoring of claim 22, wherein said flavoring is selected from a group consisting of spray-dried flavor compositions and glycerin-based flavor compositions.
